(19) United States
(12) Patent Application Publication
(10) Pub. No.: US 2020/0121780 A1
(43) Pub. Date: Apr. 23, 2020

(54) METHODS TO REDUCE THE LIKELIHOOD OF MATERNAL AND FETAL ZIKA VIRUS DISEASE

(71) Applicant: Cedars-Sinai Medical Center, Los Angeles, CA (US)

(72) Inventors: Vaithilingaraja Arumugaswami, Los Angeles, CA (US); Laura Martinez, Los Angeles, CA (US); Deisy Contreras, Monterey Park, CA (US); Gustavo Garcia, JR., Gardena, CA (US)

(73) Assignee: Cedars-Sinai Medical Center, Los Angeles, CA (US)

(21) Appl. No.: 16/605,696

(22) PCT Filed: Apr. 13, 2018
(86) PCT No.: PCT/US2018/027602

§ 371 Date: Oct. 16, 2019

Related U.S. Application Data
(60) Provisional application No. 62/490,225, filed on Apr. 26, 2017.

(51) Int. Cl. A61K 39/12; A61K 9/00; A61K 35/76; A61K 49/00

(52) U.S. Cl. CPC A61K 39/12; A61K 9/0048; A61K 2039/5254; A61K 35/76; A61K 49/0008; A61K 9/0031

(57) ABSTRACT

The present invention describes methods of eliciting protective immune response against the Zika virus. The invention also describes methods of screening for vaccine candidates.

### CROSS-REFERENCE TO RELATED APPLICATIONS

This application includes a claim of priority under 35 U.S.C. § 119(e) to U.S. provisional patent application No. 62/490,225, filed Apr. 26, 2017, the entirety of which is hereby incorporated by reference.

### FIELD OF INVENTION

This invention relates to the treatment of Zika infections.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Zika virus (ZIKV) is a major human pathogen that causes developmental abnormalities, including microcephaly and congenital eye disease. No therapies or vaccines are currently available. ZIKV is a member of the Flaviviridae family, which is comprised of other human pathogens, such as the West Nile, Dengue, and St. Louis Encephalitis viruses. Its main mode of transmission is vectorial by the mosquitoes, Aedes aegypti and A. albopictus. Currently, there have been reports of direct human-to-human infection by sexual intercourse and perinatal transmission, which can be ascribed to transplacental transmission (TPT). ZIKV RNA has been detected in placental tissue and amniotic fluid of women whose fetuses have been affected by microcephaly. Approximately 80% of adults infected with ZIKV remain asymptomatic, while the remaining infected population exhibit mild symptoms. The histopathological characteristics of ZIKV affected fetal brain lesions include neuron-shaped calcifications in the cortex, diffused astrogliosis, activated microglial cells and macrophages in the cerebrum, and mild perivascular cuffing with T-cells and some B-cell infiltrates. Cell surface protein AXL has been shown to be a key viral entry receptor to various brain cell types.

ZIKV particles contain a positive sense, single-stranded RNA genome of 10,794 bases. The genome is organized as 5′UTR-C-prM-E-NS1-NS2A-NS2B-NS3-NS4A-2K-NS4B-NS5-3′UTR, with untranslated regions (UTR) flanking a protein-coding region. The latter encodes a single polyprotein (3,419 amino acids) that is co- and post-translationally cleaved by cellular and viral proteases into about 11 larger and 2 smaller (product of Ci and 2K) peptides. The capsid, membrane, and envelope proteins form the structural components of viral particles, whereas the non-structural (NS) proteins are involved in viral genome replication and assembly. 5′UTR and 3′UTR stem loop RNA structures are critical for the initiation of viral genome translation and replication in other flaviviruses. Hybridization or base-pairing of complementary sequences from the 5′UTR and 3′UTR terminals result in RNA cyclization, which is essential for RNA replication of Flaviviruses. The 3′ UTR has been proposed to play a vital role in pathogenesis and host adaptation of Flavivirus.

ZIKV prophylactic strategies to limit and eliminate ZIKV infection are urgently needed. An aim for a potent ZIKV vaccine is to eliminate maternal infection and prevent congenital brain and ocular ZIKV diseases. Investigational inactivated and attenuated ZIKV vaccines, based on circular DNA, are currently under development. For evaluating vaccine efficacy and safety, animal model systems are critical. A recent study demonstrated that vaccination with inactivated ZIKV and adenoviral vectors carrying ZIKV envelope gene produced neutralizing antibodies and provided protection from ZIKV infection in rhesus monkeys. A recent study showed that the immunity elicited by African lineage ZIKV protects rhesus macaques against subsequent infection with Asian lineage ZIKV. Chimeric ZIKV having the PrM-E protein of DENV exhibited dual protection for ZIKV and DENV in a mouse model. The availability of a biologically relevant cell based culture system and small animal model to evaluate ZIKV vaccine potency can accelerate the pace of vaccine development efforts.

In vivo studies using mouse model systems have shed light on the causal relationship of ZIKV to placental infection and fetal microcephaly. Animal model systems can further provide understanding of ZIKV disease pathogenesis to help evaluate antiviral therapies or vaccines against ZIKV-mediated congenital disease. Multiple studies have shown that adult wild-type (WT) mice are resistant to ZIKV infection, due to a robust innate immune system that limits the establishment of active infection. ZIKV NS5 protein was unable to attenuate the innate immune system in mice by degrading STAT2, which is a mechanism used by ZIKV to establish infection in humans. Mice deficient in type I IFN receptor (ifnar1^{-/-}) signaling pathway have been established to study ZIKV pathogenesis during congenital and adult infection. ZIKV infected ifnar1^{-/-} mice show high levels of viral burden in the brain, spinal cord, and testes. Subcutaneous infection of pregnant ifnar1^{-/-} mice (E6.5 or 7.5) or WT mice treated with anti-ifnar monoclonal antibody resulted in viremia and systemic spread of ZIKV to the trophoblasts of the maternal and fetal placenta. Furthermore, female ifnar1^{-/-} mice crossed to WT males produced heterozygous fetuses that resemble the immune status of human fetuses. Vaginal exposure of ZIKV during the first trimester of pregnancy leads to fetal growth restriction and brain infection in wild-type (WT) mice, and loss of pregnancy in ifnar1^{-/-} mice. Others have shown that ZIKV replicates in the female reproductive tract for several days depending on the estrus cycle phase. The virus also infects the testes and epididymis of male mice, further decreasing levels of testosterone and oligospermia after tissue injury. Moreover, ZIKV persists in semen of mice, which may lead to male infertility.

Accordingly, there is a need in the art for methods of treating, preventing, reducing the likelihood of ZIKV infections, and reducing the detrimental impact of ZIKV infections. Further, this is also a need for methods of screening for additional treatment and vaccination options.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of eliciting an immune response in a subject in need thereof, comprising: administering Zika virus via an ocular route, rectal route, topical route and/or intradermal route to the subject and thereby eliciting an immune response. In various embodiments, the method can further comprise administering a booster dose of the Zika virus via the ocular route, rectal route, topical route and/or intradermal route to the subject and thereby eliciting an immune response.

In various embodiments, the booster dose can be administered about 7 to 14 days after the first dose of the Zika virus.

In various embodiments, the method further comprise administering an additional booster dose about 1-3 months after the first dose of the Zika virus.

In various embodiments, administering can be via the ocular route.

In various embodiments, the Zika virus can be PRAVABC59 genotype. In various embodiments, the Zika virus can be an African lineage genotype, or an Asian lineage genotype. In various embodiments, the Zika virus can be an attenuated Zika virus.

In various embodiments, the immune response can be a protective immune response.

In various embodiments, eliciting the immune response can decrease the likelihood that the subject or a fetus of the subject will be infected with the Zika virus.

In various embodiments, eliciting the immune response can decrease or reduce developmental abnormalities of a fetus of the subject.

Various embodiments of the present invention provide for a method of eliciting an immune response in a subject in need thereof, comprising: administering an attenuated Zika virus via an ocular route, rectal route, topical route, intradermal, subcutaneous, transmucosal, and/or nasal route to the subject and thereby eliciting an immune response.

In various embodiments, the method can further comprise administering a booster dose of the Zika virus via the ocular route, rectal route, topical route intradermal, subcutaneous, transmucosal, and/or nasal route to the subject and thereby eliciting an immune response.

In various embodiments, the booster dose can be administered about 2 weeks after the first dose of the Zika virus.

In various embodiments, the method can comprise administering an additional booster dose about 1-3 months after the first dose of the Zika virus.

In various embodiments, the immune response can be a protective immune response.

Various embodiments provide for a method of testing a Zika virus vaccine candidate, comprising: providing a non-human animal model; administering the Zika virus vaccine candidate to the non-human animal model; monitoring the non-human animal model for signs of Zika virus-induced disease; selecting the Zika virus vaccine candidate for further testing if the Zika virus vaccine candidate provides at least one protective effect on the non-human animal model.

In various embodiments, the non-human animal model can be a mouse model.

In various embodiments, the non-human animal model can be an ifnar1^{-/-} mouse. In various embodiments, the ifnar1^{-/-} mouse can be an A129 mouse or IFN-αβR-KO mouse.

In various embodiments, administering can be via an ocular route, rectal route, topical route and/or intradermal route.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1F shows that ZIKV infection via ocular route is non-lethal in ifnar1^{-/-} mice in accordance with various embodiments of the present invention. (1A) Schematic diagram of experimental outline is shown. (1B) Kaplan-Meier survival plot shows mortality comparison between ocular and intra-nasal routes of infection. (1C) Percent body weight change in ocular route of ZIKV-infected mice. (1D) Graph shows ZIKV genome copies in the serum of ocular route infected mice. Graphs present tissue viral load at day 3 (1E) and day 7 (1F) post ZIKV infection.

FIG. 2A-2D shows splenic response to ocular route of ZIKV infection in ifnar1^{-/-} mice in accordance with various embodiments of the present invention. (2A and 2B) ZIKV caused splenic enlargement at 7 dpi. (2C) IHC analysis of infiltrating mononuclear cells in ZIKV infected spleen. (2D) IHC analyses show that ZIKV antigen NS4B (green) present in splenic macrophages (F4/80⁺ cells in red).

FIG. 3A-3F shows that ocular immunization protects ifnar1^{-/-} mice from ZIKV challenge in accordance with various embodiments of the present invention. (3A) Kaplan-Meier survival plot shows mortality comparison between vaccinated and unvaccinated groups. Graphs show percent body weight change in unvaccinated (3B) and vaccinated (3C) mice challenged with ZIKV. (3D) Graph shows spleen weight of vaccinated mice challenged with high and low doses of ZIKV. Two-tailed unpaired, nonparametric Mann-Whitney test was conducted. (3E and 3F) Histograms show flow cytometry analysis of various lymphocyte populations in selected mice. Vaccination results in proliferation of T and B cells, and expansion of CD4⁺CD27⁺ memory T cells in the spleen.

FIG. 4A-4B shows that ZIKV infection of pregnant ifnar1^{-/-} mice (E9.5 post-conception) causes intra-uterine growth retardation of fetuses. (4A) Body weight of E18.5 fetuses of uninfected and infected pregnant mice (via intra-vaginal or subcutaneous route). (4B) Growth retardation phenotype observed in a fetus taken from a mother after ZIKV subcutaneous infection.

FIG. 5 depicts a schematic diagram of the experimental schedule used for maternal ZIKV immunization and challenge in accordance with various embodiments of the present invention.

FIG. 6A-6B depicts body weight of vaccinated and unvaccinated mice at various time points post-immunization in accordance with various embodiments of the present invention. (6A) Dot plot shows the body weight of mice in each group. Black bars indicate the means. (6B) Percent body weight change of pregnant mice in each group compared to day 31 post-immunization.

FIG. 7 depicts fetal weight and images of vaccinated and unvaccinated groups in accordance with various embodiments of the present invention.

FIG. 8A-8G shows that central memory T cell subgroups are increased during ZIKV challenge of vaccinated mice in accordance with various embodiments of the present invention. Flow cytometry analysis of spleens for percentages of T cell population at 7 days post ZIKV challenge of unvaccinated (8A), DD-vaccinated (8B) and SD-vaccinated (8C) groups representing total T cell percent populations and memory subtypes. Dot plots represent the total CD4⁺ (8D) and CD8⁺ (8E) T cells. (8F, 8G) Total T cell memory subtypes, effector (CD27⁺CD62L⁻), central (CD27⁺CD62L⁺) and naïve (CD27⁻CD62L⁺) groups. Statistical analysis was carried out using a two-tailed unpaired, non-parametric Mann-Whitney test, where *p<0.05 and **p<0.001.

FIG. 9A-9B depicts classification of memory B cell subtypes during ZIKV challenge of vaccinated mice in accordance with various embodiments of the present invention. (9A) Flow cytometry analysis of spleens 7 days post ZIKV challenge of unvaccinated, DD-vaccinated and SD-vaccinated groups. (9B) Percentages of total B cell memory subtypes, effector (CD27⁺CD62L″), central (CD27⁺CD62L⁺) and naïve (CD27⁻CD62L⁺) groups are shown in dot plot. Statistical analysis was carried out using a two-tailed unpaired, non-parametric Mann-Whitney test, where *p<0.05 and **p<0.001.

FIG. 10A-10E depicts immunophenotypic profile of antigen-presenting cell subtypes of ZIKV vaccinated and challenged mice in accordance with various embodiments of the present invention. (10A) Representative splenic flow cytometry analysis of antigen presenting cells, which includes classical dendritic cells (cDCs), plasmacytoid dendritic cells (pDCs), CD8α⁺ DCs and macrophages of unvaccinated, vaccinated double-dose (DD) and vaccinated single dose (SD) ZIKV infected mice. Numbers represent population percentages. Dot plots illustrating total percent population of cDCs (10B), CD8α⁺ DC cells (10C), pDCs (10D), and macrophages (10E). Statistical analysis was carried out using a two-tailed unpaired, non-parametric Mann-Whitney test, where *p<0.05 and **p<0.001. MNCs: Mononuclear Cells

FIG. 11A-11D depicts ZIKV infection via rectal route in ifnar1^{-/-} male mice in accordance with various embodiments of the present invention. (11A) Percent body weight change in PBS control and ZIKV-infected mice via rectal route of infection. ZIKV was inoculated at 3.4×10⁶ pfu per mouse in 20 μl volume. (11B) Graph shows ZIKV genome copies in the serum of rectal route infected mice. Two-tailed unpaired, nonparametric Mann-Whitney test was conducted. (11C and 11D) ZIKV infection caused splenic enlargement at 7 dpi.

FIG. 12A-12D shows ZIKV infection induces splenomegaly and triggers an effector T cell response in the spleen after topical route of infection in accordance with various embodiments of the present invention. (12A) Graph represents percent body weight of control and ZIKV-infected mice for 3 and 7 dpi post-topical route of infection (12B) Bar graph illustrating total spleen weight of control (PBS) and ZIKV infected mice at 7 dpi. Error bars represent standard deviation (SD). (12C) Gross image of spleens from PBS and ZIKV infected mice at 7 dpi. (12D) Flow cytometric analyses of spleens from PBS and ZIKV mice showing B and T cell subpopulations at 7 dpi. Numbers indicate cell population percentage.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

“Topical” administration as used herein includes inoculation or application of the therapeutic composition (e.g., vaccine, virus, attenuated virus) on the surface of the skin.

“Intradermal” administration as used herein includes inoculation or injection into the dermis layer below the epidermis layer of skin. Intradermal as used herein does not include subcutaneous.

“Subcutaneous” administration as used herein includes inoculation or injection below the dermis and epidermis layers of the skin.

One skilled in the art will recognize many methods and materials similar or equivalent to those described herein, which could be used in the practice of the present invention. Indeed, the present invention is in no way limited to the methods and materials described.

One distinctive teratogenic outcome of ZIKV infection is its effect on fetal ocular development in infected mothers. Babies born with microcephaly displayed macular pigment mottling and loss of foveal reflex, with at least one severe case showing macular atrophy. These virus-related eye defects occur during the first or second trimester of fetal development in infected pregnant women. In adult ifnar1^{-/-} mice, ZIKV infects specific target cells in different regions of the eye, such as the iris, retina, and optic nerve, further causing conjunctivitis, pan-uveitis, and neuroretinitis. However, eye pathology was not detected in congenitally infected ifnar1^{-/-} fetuses from C57BL/6 ifnar1^{-/-} dams. ZIKV induced congenital ocular disease is still not well understood. Our study using human fetal retinal pigment epithelial (FRPE) cells revealed that these cells are highly susceptible to ZIKV infection, further resulting in apoptotic cell death and activation of host immune and inflammatory genes.

Both innate and specific immune responses are critical for controlling viral infections. Upon virus infection, the cells can detect pathogen associated molecular patterns (PAMPs), such as viral RNA, by toll-like receptors to activate innate immune and inflammatory pathways through NF-kappa β and interferon (IFN)-stimulated genes. The resulting activated host effector factors may limit infection by directly inhibiting the pathogen or inducing apoptotic cell death. Moreover, specific humoral and T-cell mediated immune responses can further limit viral replication. The development of a vaccine is a global health priority, yet our knowledge of immune protection against ZIKV is limited.

ZIKV infection in adults is mostly mild in nature, however, fetal infection results in death of ocular and neural progenitor cells. Immunization of women prior to pregnancy can limit maternal infection upon exposure and protect the developing fetus from severe developmental defects.

Various embodiments of the invention provide for a method of eliciting an immune response in a subject in need thereof, comprising: administering Zika virus via an ocular route, rectal route, topical route and/or intradermal route to the subject and thereby eliciting an immune response. In further embodiments, the method further comprises administering a booster dose of the Zika virus via an ocular route, rectal route, topical route and/or intradermal route to the subject and thereby eliciting an immune response.

In various embodiments, the booster dose is administered about 7-14 days after the first administration of the Zika virus. For example, the booster dose can be administered 10 days after the first administration of the Zika virus. In various embodiments, the booster dose can be administered about 2-6 weeks after the first administration of the Zika virus. For example, the booster dose can be administered about 4 weeks after first administration of the Zika virus.

In various embodiments, more than one booster dose is administered. In particular embodiments, a first booster dose can be administered about 7-14 days after the first administration of the Zika virus, and an additional booster dose can be administered about 1-3 months after the first administration of the Zika virus.

In still other embodiments, additional booster doses (e.g., more than 2 booster dose) can be administered; for example, an additional booster dose can be administered every 1, 5, or 10 years after the first administration of the Zika virus.

In still other embodiments, a booster dose can be administered before, during or after a high risk event. For example, in a subject previously administered the Zika virus in accordance to the present invention, a booster dose can be administered before a subject travels to a region where Zika transmission is prevalent; for example, wherein Aedes aegypti and/or A. albopictus mosquitos are prevalent. In another example, in a female subject previously administered the Zika virus for reasons related to pregnancy, a booster dose can be administered, prior or during a subsequent pregnancy.

In various embodiments, the virus is administered via the ocular route. Via the ocular route, they may be; for example, in the form of eye drops or a gel.

In various embodiments, the virus is an Asian genotype Zika virus; for example, the Asian genotype Zika virus that corresponds to GenBank accession number KU501215 (clinical isolate PRVABC59). Other Asian genotype Zika viruses can be used as well.

In other embodiments, the virus is an African genotype Zika virus including, but not limited to the East African and West African sub-types.

Additional examples of Zika virus strains include but are not limited: a Zika virus that corresponds to GenBank accession nos.: KU321639 (Brazil 2015 SPH2015), KJ776791 (French Polynesia H/PF/2013), KF383115 (Central African Republic ARB1362), KF383116 (Senegal 1968 ArD7117), KF383117 (Senegal 1997 ArD128000), KF383118 (Senegal 2001 ArD157995), KF383119 (Senegal 2001 ArD158084), KF268948 (CAR 1979 ARB13565), KF268949 (CAR 1980 ARB15076), KF268950 (CAR 1976 ARB7701), EU545988 (Yap 2007), KF993678 (Thailand 2013 PLCal ZV), JN860885 (Cambodia 2010 FSS13025), HQ234499 (Malaysia 1966 P6-740), HQ234501 (Senegal 1984 ArD41519), HQ234500 (Nigeria 1968 IbH 30656), LC002520 (Uganda 1947 MR766), KU501215 (Puerto Rico PRVABC59), KU501216 (Guatemala 8375), or KU501217 (Guatemala 103344). (Lanciotti R S, Lambert A J, Holodniy M, Saavedra S, del Carmen Castillo Signor L. Phylogeny of Zika virus in Western Hemisphere, 2015 [letter]. Emerg Infect Dis. 2016 May. Available at www.dx.doi.org/10.3201/eid2205.160065, accessed on Aug. 17, 2016.)

Still further examples of Zika viruses include but are not limited to a Zika virus selected from Table 1 below. (Arunachalam Ramaiah, Lei Dai, Deisy Contreras, Sanjeev Sinha, Ren Sun, Vaithilingaraja Arumugaswami, Comparative analysis of protein evolution and RNA structural changes in the genome of pre-epidemic and epidemic Zika virus (2016) bioRxiv.)

TABLE 1
Zika viruses (1947-2016).

| | Accession | | Year of | | |
|---|---|---|---|---|---|
| Strain name | number | Host | isolation | Country | Genotype |
| MR 766 | AY632535 | Sentinel | 1947 | Uganda | African lineage |
| | | monkey | | | |
| MR 766 | DQ859059 | Sentinel | 1947 | Uganda | African lineage |
| | | monkey | | | |
| MR_766 | HQ234498 | Sentinel | 1947 | Uganda | African lineage |
| | | rhesus | | | |
| IbH_30656 | HQ234500 | Homo sapiens | 1968 | Nigeria | African lineage |
| ArD_41519 | HQ234501 | Aedes | 1984 | Senegal | African lineage |
| | | africanus | | | |
| ARB13565 | KF268948 | Aedes | 1976 | CAR | African lineage |
| | | africanus | | | |
| ARB15076 | KF268949 | Aedes opok | 1980 | CAR | African lineage |
| ARB7701 | KF268950 | Aedes | 1976 | CAR | African lineage |
| | | africanus | | | |
| ArB1362 | KF383115 | Aedes | 1968 | CAR | African lineage |
| | | africanus | | | |
| ArD7117 | KF383116 | Aedes | 1968 | Senegal | African lineage |
| | | luteocephalus | | | |
| ArD128000 | KF383117 | Aedes | 1997 | Senegal | African lineage |
| | | luteocephalus | | | |
| ArD157995 | KF383118 | Aedes dalzieli | 2001 | Senegal | African lineage |
| ArD158084 | KF383119 | Aedes dalzieli | 2001 | Senegal | African lineage |
| ArD142623 | KF383120 | Anopheles | 2000 | Senegal | African lineage |
| | | coustani | | | |
| MR 766 | KU720415 | Sentinel | 1947 | Uganda | African lineage |
| | | monkey | | | |
| MR766-NIID | LC002520 | Sentinel | 1947 | Uganda | African lineage |
| | | monkey | | | |
| MR 766 | NC_012532 | Sentinel | 1947 | Uganda | African lineage |
| | | monkey | | | |
| ArD158095 | KF383121 | | | | African lineage |
| EC_Yap | EU545988 | Homo sapiens | 2007 | Micronesia | Asian lineage |
| P6-740 | HQ234499 | Aedes aegypti | 1966 | Malaysia | Asian lineage |
| FSS13025 | JN860885 | Homo sapiens | 2010 | Cambodia | Asian lineage |
| PLCal_ZV | KF993678 | Homo sapiens | 2013 | Canada | Asian lineage |
| H/PF/2013 | KJ776791 | Homo sapiens | 2013 | French | Asian lineage |
| | | | | Polynesia | |
| Z1106033 | KU312312 | Homo sapiens | 2015 | Suriname | Asian lineage |
| ZikaSPH2015 | KU321639 | Homo sapiens | 2015 | Brazil | Asian lineage |
| BeH818995 | KU365777 | Homo sapiens | 2015 | Brazil | Asian lineage |
| BeH819015 | KU365778 | Homo sapiens | 2015 | Brazil | Asian lineage |
| BeH819966 | KU365779 | Homo sapiens | 2015 | Brazil | Asian lineage |
| BeH815744 | KU365780 | Homo sapiens | 2015 | Brazil | Asian lineage |
| Brazil-ZKV2015 | KU497555 | Homo sapiens | 2015 | Brazil | Asian lineage |
| PRVABC59 | KU501215 | Homo sapiens | 2015 | Puerto | Asian lineage |
| | | | | Rico | |
| 103344 | KU501216 | Homo sapiens | 2015 | Guatemala | Asian lineage |
| 8375 | KU501217 | Homo sapiens | 2015 | Guatemala | Asian lineage |
| Haiti/1225/2014 | KU509998 | Homo sapiens | 2014 | Haiti | Asian lineage |
| Natal RGN | KU527068 | Homo sapiens | 2015 | Brazil | Asian lineage |
| SSABR1 | KU707826 | Homo sapiens | 2015 | Brazil | Asian lineage |
| VE_Ganxian | KU744693 | Homo sapiens | 2016 | China | Asian lineage |
| GDZ16001 | KU761564 | Homo sapiens | 2016 | China | Asian lineage |
| MRS_OPY_Martin | KU647676 | Homo sapiens | 2015 | Martinique | Asian lineage |
| ique_PaRi_2015 | | | | | |
| tc/THA/2014/SVO1 | KU681081 | Homo sapiens | 2014 | Thailand | Asian lineage |
| 27-14 | | | | | |
| tc/PHL/2012/CPC- | KU681082 | Homo sapiens | 2012 | Philippines | Asian lineage |
| 0740 | | | | | |
| BeH823339 | KU729217 | Homo sapiens | 2015 | Brazil | Asian lineage |
| BeH828305 | KU729218 | Homo sapiens | 2015 | Brazil | Asian lineage |
| GD01 | KU740184 | Homo sapiens | 2016 | China | Asian lineage |
| FLR | KU820897 | Homo sapiens | 2015 | Colombia | Asian lineage |
| ZJO3 | KU820899 | Homo sapiens | 2016 | China | Asian lineage |

In various embodiments, the Zika virus is an attenuated Zika virus.

In various embodiments, the immune response elicited by the method is a protective immune response. For example, eliciting the immune response decreases the likelihood that the subject or a fetus of the subject will be infected with the Zika virus. In various embodiments, eliciting the immune response reduces developmental abnormalities of a fetus of the subject. In various embodiments, eliciting the immune response reduces the likelihood of the fetus and baby having microcephaly. In various embodiments, eliciting the immune response reduces the extent of microcephaly of the fetus and baby. Signs and symptoms of microcephaly can include a smaller than normal head circumference that usually remains smaller than normal as the child grows, dwarfism or short stature, delayed motor and speech functions, mental retardation, seizures, facial distortions, hyperactivity, balance and coordination problems, and other brain-related or neurological problems. Thus, reducing the extent of microcephaly can include reducing the number of signs and symptoms, or it can include reducing the severity of the signs and symptoms of microcephaly.

In various embodiments, the subject is a male. In other embodiments, the subject is a female. In particular embodiments, the subject is a pregnant female. In other embodiments, the subject is a female planning on becoming pregnant. In other particular embodiments, the subject is a person living in or traveling to a region where Zika transmission is prevalent; for example, wherein Aedes aegypti and/or A. albopictus mosquitos are prevalent.

Various embodiments provide for a method of testing a Zika virus vaccine candidate, comprising: providing a non-human animal model; administering the Zika virus vaccine candidate to the non-human animal model; monitoring the non-human animal model for signs of Zika virus-induced disease; selecting the Zika virus vaccine candidate for further testing if the Zika virus vaccine candidate provides at least one protective effect on the non-human animal model. Examples of a sign of Zika virus-induced disease include but are not limited to changes in body weight, lethargy, neurological disease signs (e.g., incoordination of body movement—ataxia, hemiplegia, paraplegia), viral load in tissue and/or blood. Protective effects include but are not limited prevention or reduction of a sign of Zika virus-induced disease.

In various embodiments, the non-human animal model is a mouse model. In various embodiments, the non-human animal model is ifnar1^{-/-} mouse. In various embodiments, the ifnar1^{-/-} mouse is an A129 mouse or IFN-αβR-KO mouse. Additional examples of non-human animal model include but are not limited to ISG15 KO, IFN-αβR-Ragy KO.

In various embodiments, administering to the model is via an ocular route, rectal route, topical, and/or intradermal route.

In various embodiments, the pharmaceutical compositions comprising a Zika virus according to various embodiments of the invention may be formulated for delivery via certain routes of administration. “Route of administration” may refer to any administration pathway that will elicit a protective immune response, including but not limited to ocular, rectal, topical, and intradermal. Additional pathways include transdermal and transmucosal. In some embodiments, a nasal route or subcutaneous route may be used; for example, when it is an attenuated or inactivated Zika virus. “Transdermal” administration may be accomplished using a topical cream or ointment or by means of a transdermal patch. Via the ocular route, they may be in the form of eye drops or in a gel.

The compositions may be in the form of solutions or suspensions for infusion or for injection, or as lyophilized powders, tablets, gel capsules, sugar-coated tablets, syrups, suspensions, solutions, powders, granules, emulsions, microspheres or nanospheres or lipid vesicles or polymer vesicles allowing controlled release, solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective Zika virus can be indicated to the skilled artisan by the in vitro responses or responses in animal models. Examples of dosages used per mouse include 10 copies to 10e8 pfu dose per mouse per dose; for example, 10 copies, 10e2, 10e3, 10e4, 10e5, 10e6, 10e7, 10e8 pfu dose. These doses can be given in two to three doses. Such dosages typically can be reduced by up to about one order of magnitude in concentration or amount without losing the relevant biological activity. Thus, the actual dosage can depend upon the judgment of the physician, the condition of the patient, and the effectiveness of the therapeutic method based, for example, on the in vitro responsiveness of the relevant primary cultured cells or histocultured tissue sample, such as biopsied infected tissues, or the responses observed in the appropriate animal models, as previously described.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Mouse Model to Investigate ZIKV Disease Pathogenesis

We utilized ifnar1^{-/-} (A129 or IFN-αβR-KO) mice (MMRRC at Jackson Laboratory) for infection studies. For the initial experiment, mice (n=4 per group) were anesthetized with isoflurane in a desiccator jar within a ductless fume hood. Ear tags were applied for identification purposes. Forty microliters of PRVABC59 ZIKV infectious inoculum (2×10⁶ pfu/mouse) was injected subcutaneously (SC) into each of the right and left hind legs just above the ankle inside a biosafety cabinet. Control mice were inoculated with 40 μL phosphate buffered saline (PBS) in each of the hind legs. For safety reasons, the exposed mice were housed in a dedicated infectious agent isolation cubicle room. The mice were monitored daily for body condition and neurological symptoms. Body weights were measured before euthanasia at the indicated timepoints, as well as for animals requiring euthanasia due to ZIKV-induced disease. By 6-7 dpi, the infected animals acutely reached the humane endpoints triggering euthanasia. Interestingly, mice exhibited neurological signs of posterior paralysis at 7 dpi. ZIKV was detected in the serum and various tissues. Infected brain had higher level of inflammatory gene activity.

### ZIKV Infection Through Ocular Exposure

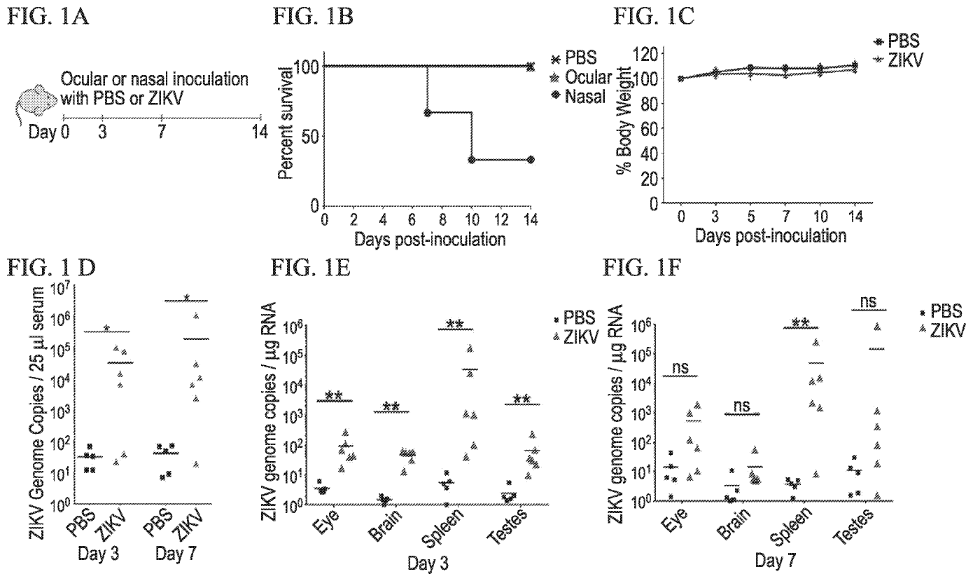

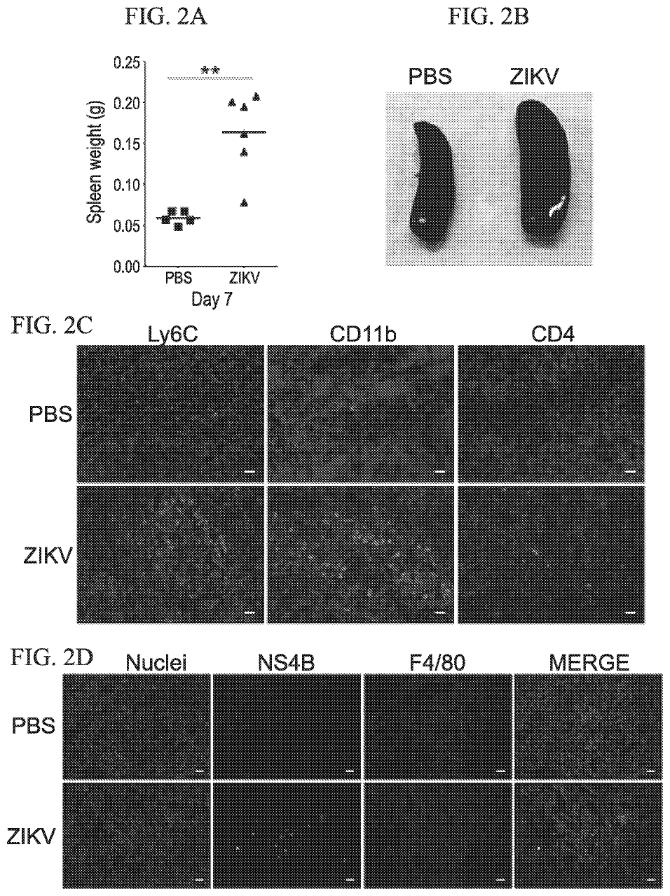

ZIKV can spread to the eye through hematogenous route or ocular exposure from infected amniotic fluid in utero. We observed that subcutaneous ZIKV infection can disseminate to the eye through blood. We then tested the possibility of ZIKV transmission through ocular route. 4-5-week-old ifnar1^{-/-} mice were used (PBS group n=5; ZIKV group n=6) (FIG. 1). PRVABC59 ZIKV ocular inoculation was performed under isoflurane anesthesia in a biological safety cabinet. 2 μl of inoculum (PBS or ZIKV at 6.8×10⁵ pfu) was applied on each ocular surface using a pipette. The entry of inoculum into each inner eye was monitored and each eyelid was closed gently for a minute to make sure the inoculum was completely absorbed. We also tested a respiratory route of ZIKV infection. For this route, ZIKV (6.8×10⁵ pfu per mouse in 20 μl) was inoculated intra-nasally under isoflurane anesthesia. Mice were monitored for 14 days for signs of neurological disease, dehydration, and morbidity. Body weight was measured at the indicated timepoints. Mice were humanely euthanized using CO₂. We observed that ocular exposed mice remained healthy, whereas nasally infected mice suffered from posterior paralysis and immobility, further requiring euthanasia at days 7 and 10 (FIG. 1A). No statistically significant change in the body weight of ocular inoculated mice was noted (FIG. 1B). In the ocular group, viremia was observed at 3 and 7 dpi suggesting establishment of active virus infection without any overt disease (FIG. 1C). Interestingly, the spleen of infected mice showed enlargement as compared to PBS inoculated mice (FIG. 2A, 2B), and our preliminary immunohistochemistry study confirmed the proliferation of splenic mononuclear cells (Ly6C, CD1 lb and CD4) (FIG. 2C) Splenic macrophages are the target cells infected by ZIKV (FIG. 2D).

We believe that an ocular route of ZIKV infection can prime the immune system and induce a strong immune response due to specialized eye barriers. Corneal epithelial cells, ocular macrophages, Langerhans cells, and cytokines may play important roles in activating and generating specific T- and B-cell responses.

ZIKV Immunization Through Ocular Route Protects Adult Mice from Lethal Subcutaneous ZIKV Challenge

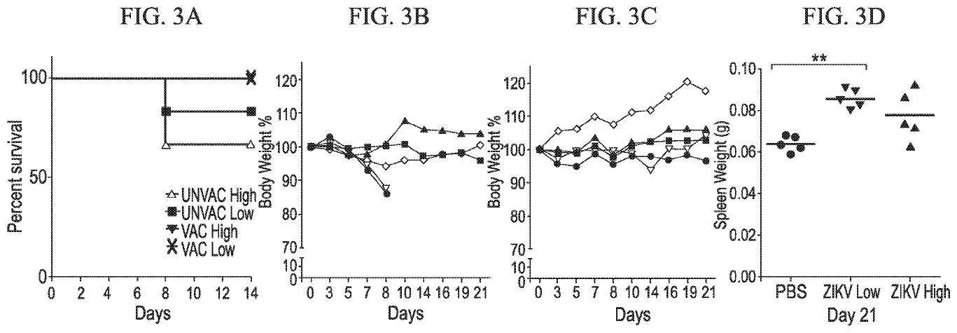

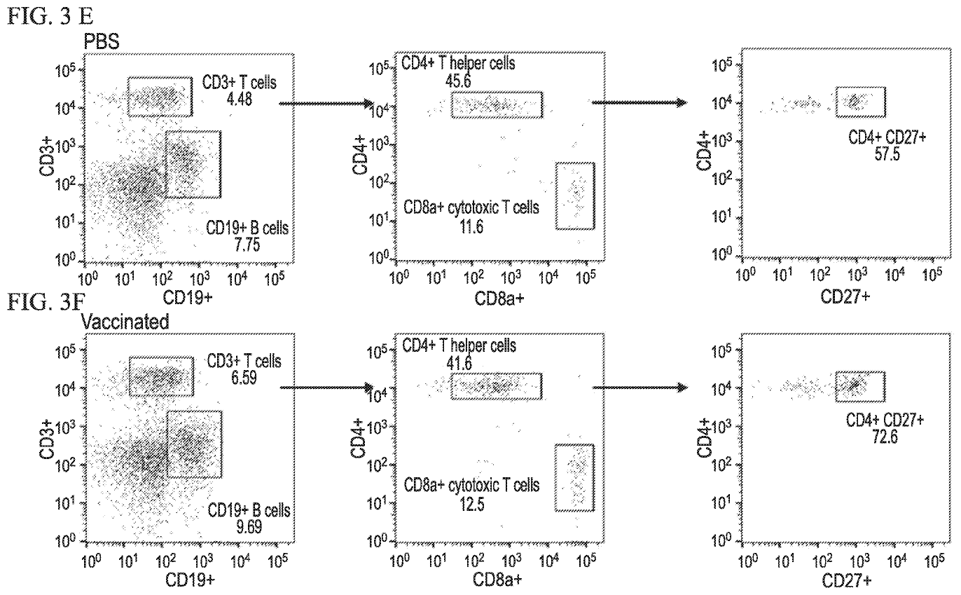

Five-week-old friar mice were immunized with ZIKV through ocular route. Unvaccinated mice received PBS only. Ten days later, both the vaccinated and unvaccinated mice challenged with ZIKV via subcutaneous route. For challenge, low (10³ pfu) and high (10⁶ pfu) doses were used. Animals were monitored for an additional 21 days. We observed that all the vaccinated mice were protected, whereas unvaccinated mice lost weight and developed posterior paralysis after 8 days of challenge with ZIKV, triggering euthanasia (FIG. 3).

ZIKV Immunization Through Ocular Route Protects Mother and Fetus from ZIKV Disease

We determined that ocular route of ZIKV infection elicits protective immunity against lethal subcutaneous ZIKV infection in ifnar1^{-/-} mice. This type I IFN independent protective effect is possibly due to induction of a potent adaptive immune response.

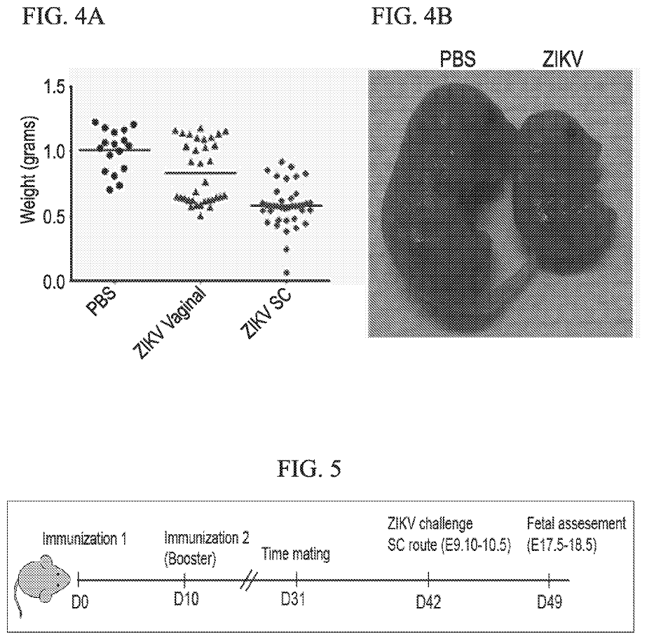

Eye and brain development begins around E8.5 in mice and ZIKV infection during this period would interfere with proper organ formation by inducing cell death. Studies show that ZIKV infection of pregnant mice caused intra-uterine growth retardation of the fetus. Our study comparing the ZIKV effect on developing mouse fetus through subcutaneous and intravaginal routes of infection showed marked reduction in fetal growth and body weight (FIG. 4).

After establishing the ZIKV maternal-fetal infection model, we set out to study the protective effect of ocular immunization in the fetal development aspect. The experimental schedule used for ZIKV immunization and challenge is shown in FIG. 5.

### Example 2

Methods: At day 0, breeding female ifnar1^{-/-} mice was immunized with ZIKV (6.8×10⁵ pfu/mouse) through ocular route (double dose of vaccination group). PBS inoculated mice was included as the unvaccinated group. We used n=6 mice per group. For booster immunization, a second ocular inoculation was performed at day 10 post-immunization. At day 10, ocular immunization was performed for single dose vaccination group as well. At day 31, time mating (timed pregnancy) was set up. At E9.5-E10.5 post-conception (day 42 post-immunization), both vaccinated (single and double dose) and unvaccinated groups were challenged with ZIKV (1×10⁶ pfu/mouse) through SC route. The mice were monitored for the next 7 days for evaluating neurological signs, weight loss, and mortality. At day 49 (E17.5-18.5), mice were humanely euthanized and fetuses were collected and weighed.

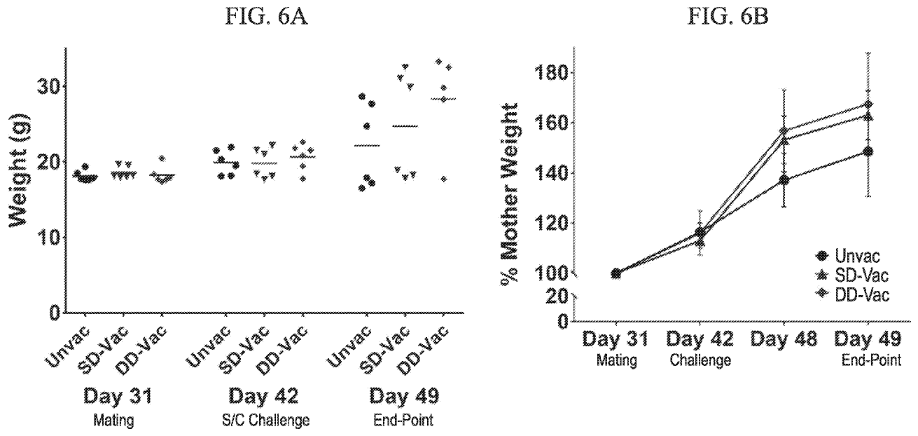

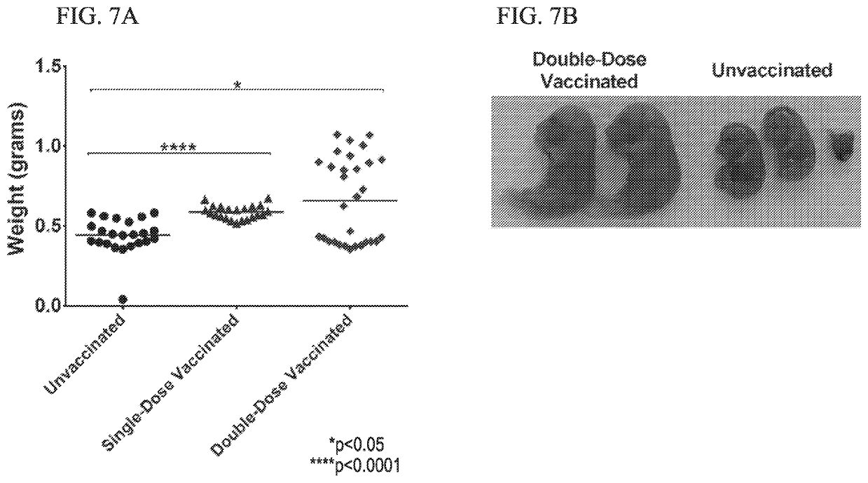

Results: We did not observe any adverse health effect of ocular vaccination in both single and double dose groups. At 31 days post-immunization 1, mice in all the groups had relatively similar body weight (FIG. 6A). At day 42 based on body weight gain, we observed n=4, n=3, and n=5 possible pregnant mice in unvaccinated, single dose (SD) and double dose (DD) groups, respectively. At day 48, one pregnant mouse in the double dose vaccinated group was euthanized to observe the fetus size. Interestingly, at day 49, only 3 mice showed signs of pregnancy (bulged abdominal cavity and weight gain) in the unvaccinated group (PBS only) suggesting pregnancy loss or pseudo-pregnancy in one of the mice. Also in this group, one non-pregnant mouse developed partial hind limb (right) paralysis. All the mice in each group were euthanized at day 49. The unvaccinated pregnant mice showed lower body weight than vaccinated groups (FIG. 6B).

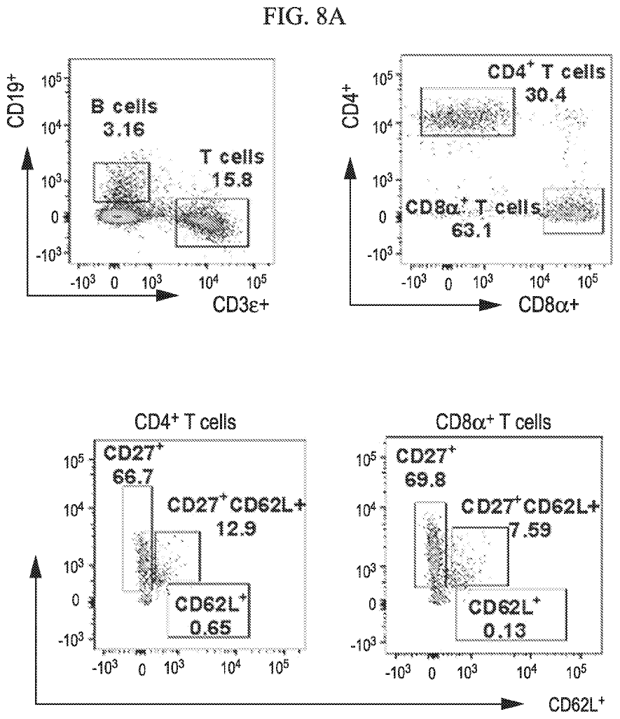

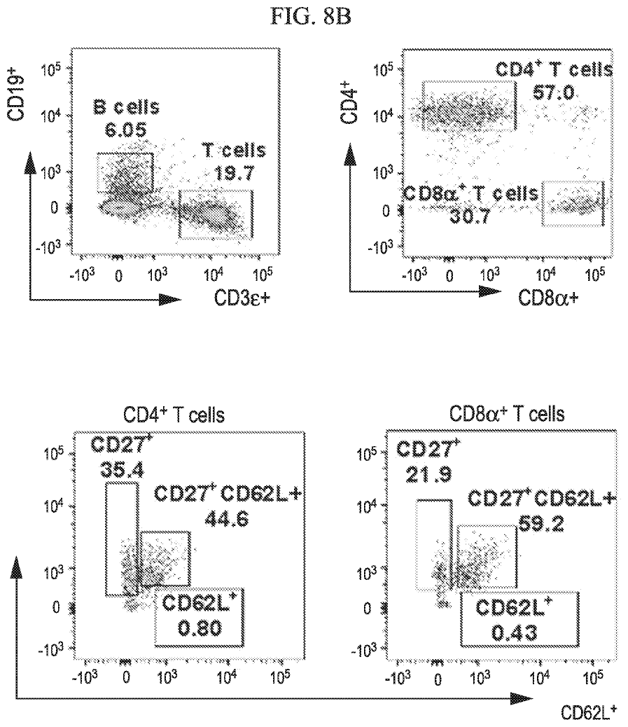

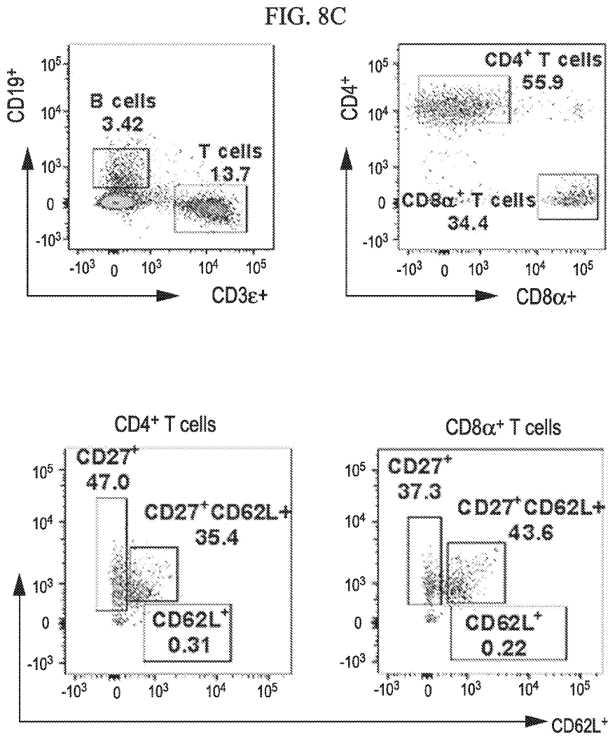

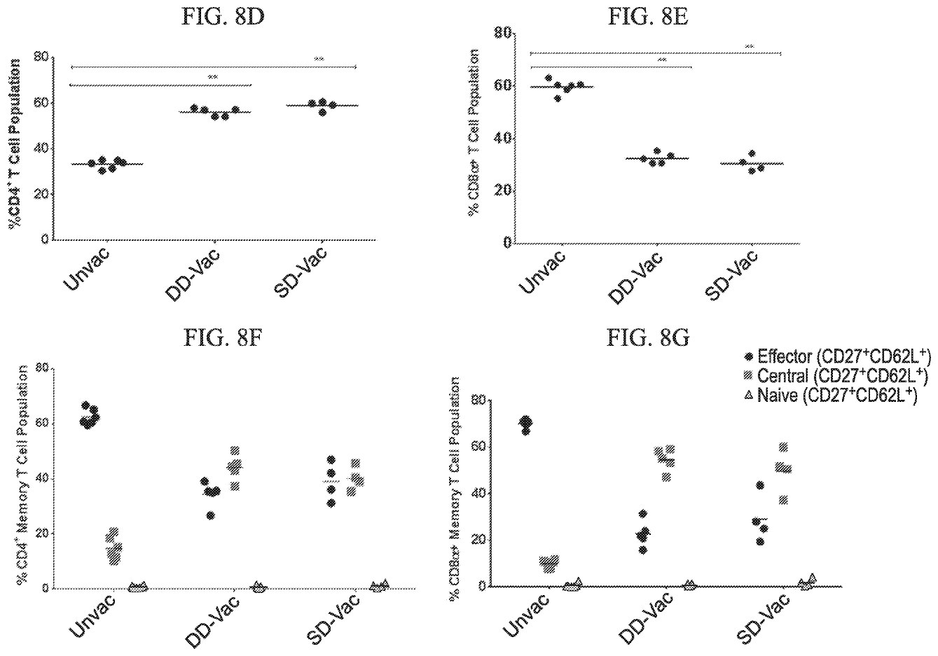

We observed that the fetuses of the unvaccinated group had significantly lowered body weight compared to the single or double dose vaccinated groups (FIG. 8A). Also it is interesting to note that an unvaccinated mouse had a partially developed fetus (FIG. 8B).

These results show that ocular route of immunization provides both maternal and fetal protection against Zika virus disease.

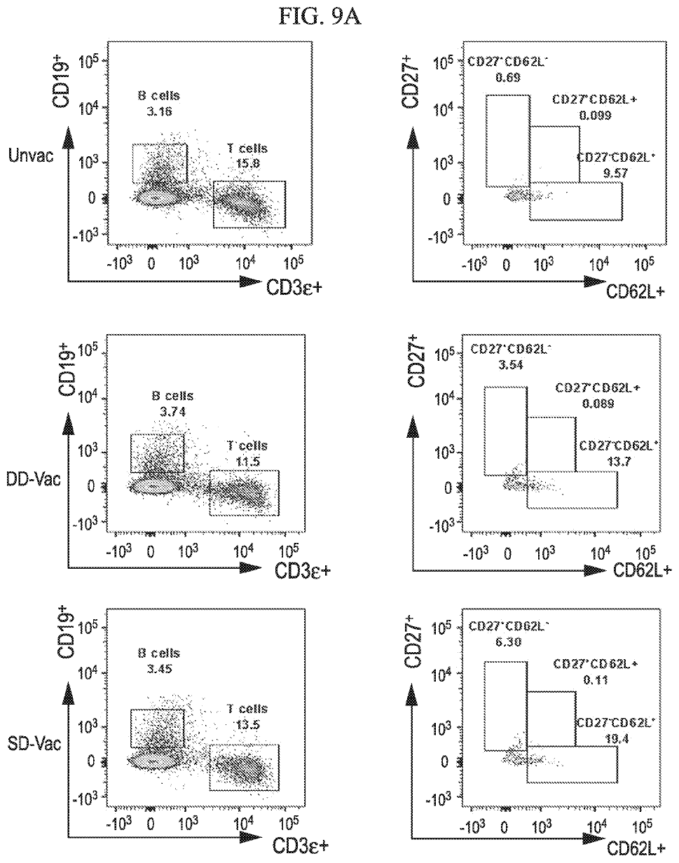

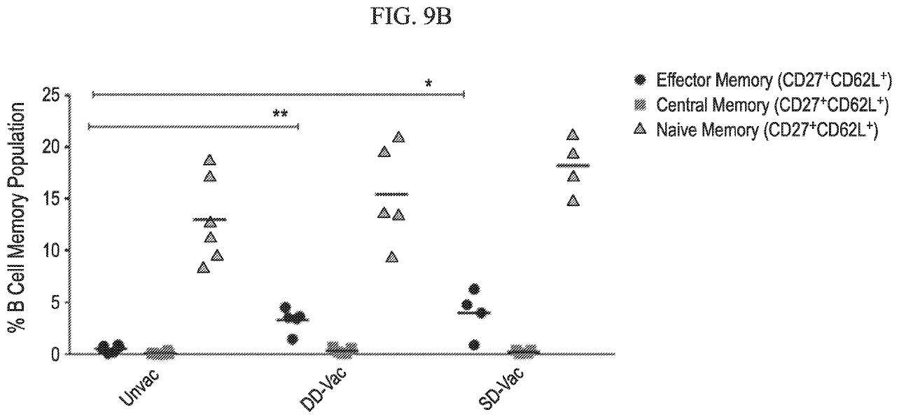

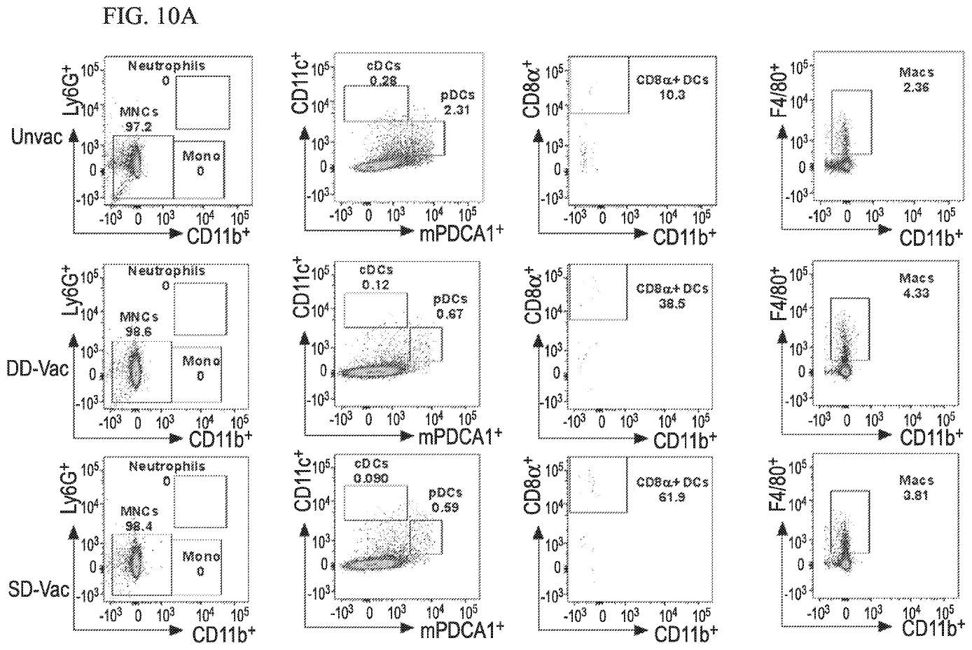

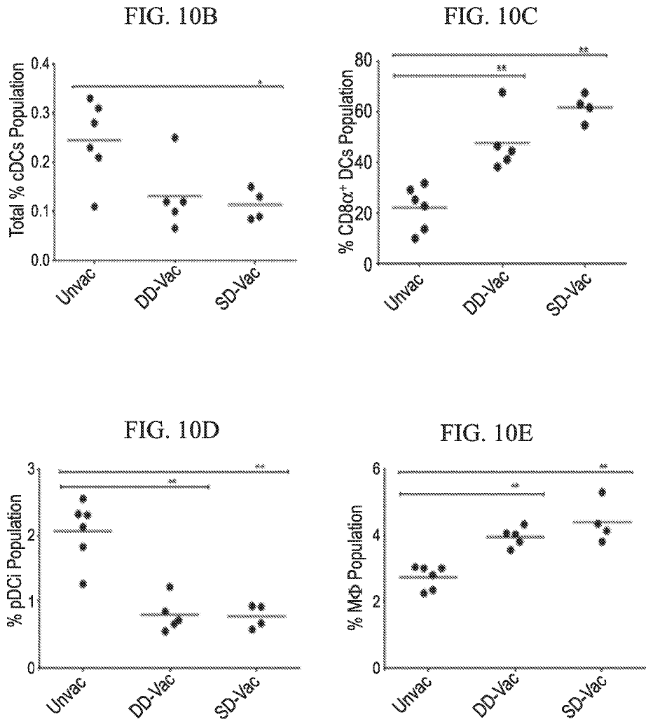

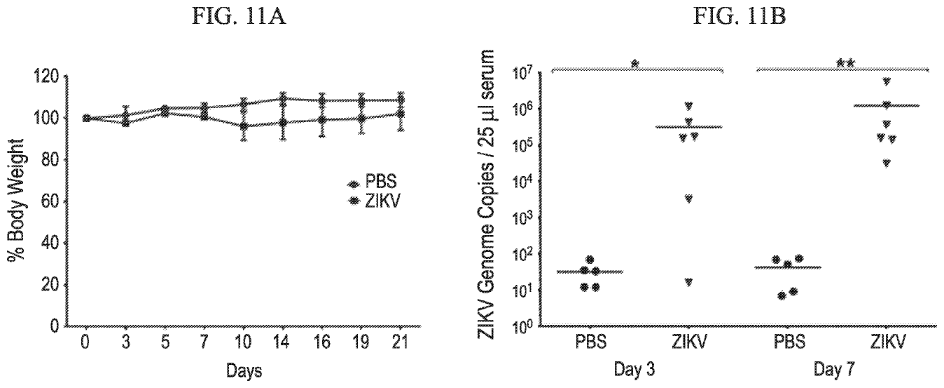

We further evaluated the immune cellular responses elicited by ZIKV vaccination and challenge. Cells harvested from spleens of ZIKV challenged unvaccinated and vaccinated (SD and DD) mice were subjected to immunostaining and flowcytometry analysis (FIGS. 8, 9 and 10). In vaccinated mice, higher CD4⁺ Th cell response was observed after ZIKV challenge, whereas in unvaccinated mice higher CD8⁺ cytotoxic T cell response was seen (FIG. 8). Vaccination also increased central memory cell populations for both Th (CD4⁺ CD27⁺ CD62L⁺) and cytotoxic (CD8⁺ CD27⁺ CD62L⁺) subtypes. Moreover, effector memory B cells (CD19⁺ CD27⁺ CD62L⁻) were increased in vaccinated group (FIG. 9). Vaccination also increased the level of antigen presenting cells CD8α±dendritic (DCs) and macrophages (FIG. 10). Adaptive memory response induced by vaccination is responsible for the observed protection.

### Example 3

### Evaluating Rectal Route of Zika Virus Infection in Ifnar1^{-/-} Mice as an Additional Route of Immunization.

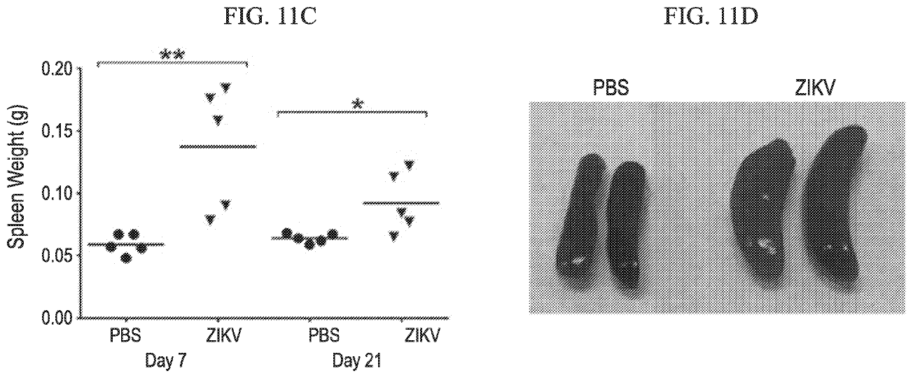

Sexual transmission of ZIKV via vaginal route is well established. Intravaginal inoculation of ZIKV in pregnant ifnar1^{-/-} mice causes intra-uterine growth retardation of fetuses and loss of pregnancy. Development of a prophylactic strategy to induce a mucosal immune response to neutralize Zika viral particles in the vaginal passage is important to prevent congenital infection. Rectal immunization against Rotavirus and Hepatitis A virus has been shown to elicit strong systemic and mucosal immune responses. Rectal infection of ifnar1^{-/-} mice with PRVABC59 did not cause mortality, however, viremia and splenomegaly were present (FIG. 11). While not wishing to be bound by any particular theory, we believe that the mucosal immune response induced by rectal immunization with ZIKV can provide anti-ZIKV protection in the reproductive mucosa. The lymphoid cells present in the rectal submucosa would respond to initial exposure to ZIKV antigen and mount an effective immune response.

While not wishing to be bound by any particular theory, we believe that the rectal route of immunization with either Asian or African genotype ZIKV of unmodified wild-type or genetically modified attenuated strains provides complete protection irrespective of age, sex, and pregnancy state.

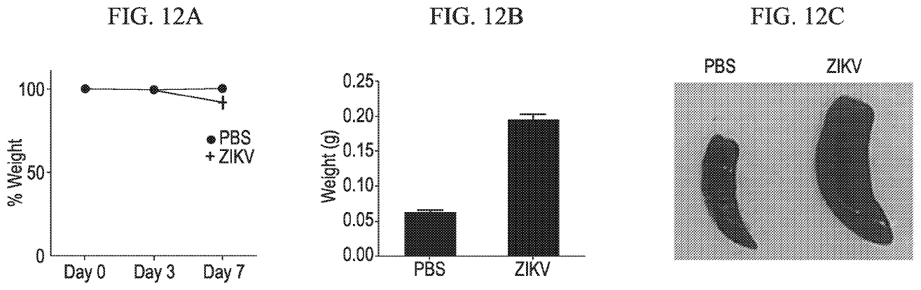

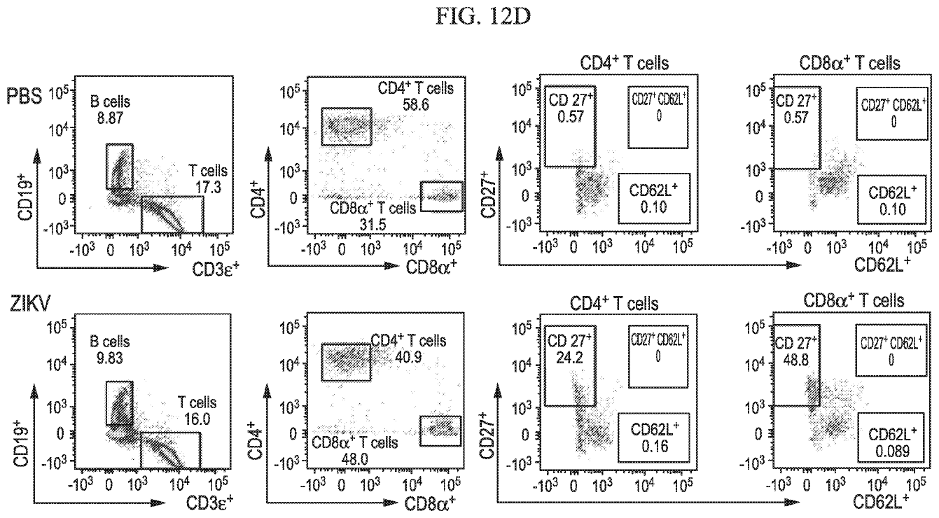

During ocular immunization, the corneal epithelium is exposed to ZIKV. The outer layer of the cornea is comprised of stratified squamous epithelial cells, which is similar to skin epithelium. To evaluate the immune response induced by topical inoculation of ZIKV, we carried out animal experiment using ifnar1^{-/-} mice. ZIKV (1×10⁶ pfu/mouse) inoculum was applied on the skin surface of the back of the mice. For this the hair on the mouse back was removed and the surface was disinfected and washed with PBS before applying ZIKV. The animals did not exhibit any neurological disease phenotype during the experimental period and were active. At 7 day post infection, the mice were humanely euthanized and the spleen tissues were collected for immune cell analysis by flowcytometry. We observed that the spleen was enlarged in infected animal (FIGS. 12B and 12C) indicating a cellular response. Flowcytometry analysis showed that ZIKV infected mice had higher level of cytotoxic CD8 T cells and effector memory T cells (CD4⁺ CD27⁺ CD62L⁻ and CD8⁺ CD27⁺ CD62L⁻) compared to uninfected mice (FIG. 12D). Taken together topical ZIKV induces adaptive immune response. We believe that topical (skin) and intradermal routes of ZIKV immunization with or without a booster dose will provide protection against ZIKV disease.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

What is claimed is:

1. A method of eliciting an immune response in a subject in need thereof, comprising:
administering Zika virus via an ocular route, rectal route, topical route and/or intradermal route to the subject and thereby eliciting an immune response.

2. The method of claim 1, further comprising administering a booster dose of the Zika virus via the ocular route, rectal route, topical route and/or intradermal route to the subject and thereby eliciting an immune response.

3. The method of claim 2, wherein the booster dose is administered about 7 to 14 days after the first dose of the Zika virus.

4. The method of claim 2, further comprising an additional booster dose about 1-3 months after the first dose of the Zika virus.

5. The method of claim 1, wherein administering is via the ocular route.

6. The method of claim 1, wherein the Zika virus is PRAVABC59 genotype.

7. The method of claim 1, wherein the Zika virus is an African lineage genotype, or an Asian lineage genotype.

8. The method of claim 1, wherein the Zika virus is an attenuated Zika virus.

9. The method of claim 1, wherein the immune response is a protective immune response.

10. The method of claim 1, wherein eliciting the immune response decreases the likelihood that the subject or a fetus of the subject will be infected with the Zika virus.

11. The method of claim 1, wherein eliciting the immune response decreases or reduces developmental abnormalities of a fetus of the subject.

12. A method of eliciting an immune response in a subject in need thereof, comprising:
administering an attenuated Zika virus via an ocular route, rectal route, topical route, intradermal, subcutaneous, transmucosal, and/or nasal route to the subject and thereby eliciting an immune response.

13. The method of claim 12, further comprising administering a booster dose of the Zika virus via the ocular route, rectal route, topical route intradermal, subcutaneous, transmucosal, and/or nasal route to the subject and thereby eliciting an immune response.

14. The method of claim 13, wherein the booster dose is administered about 2 weeks after the first dose of the Zika virus.

15. The method of claim 13, further comprising an additional booster dose about 1-3 months after the first dose of the Zika virus.

16. The method of claim 12, wherein the immune response is a protective immune response.

17. A method of testing a Zika virus vaccine candidate, comprising:
providing a non-human animal model;
administering the Zika virus vaccine candidate to the non-human animal model;
monitoring the non-human animal model for signs of Zika virus-induced disease;
selecting the Zika virus vaccine candidate for further testing if the Zika virus vaccine candidate provides at least one protective effect on the non-human animal model.

18. The method of claim 17, wherein the non-human animal model is a mouse model.

19. The method of claim 17, wherein the non-human animal model is ifnar1^{-/-} mouse.

20. The method of claim 19, wherein the ifnar1^{-/-} mouse is an A129 mouse or IFN-αβR-KO mouse.

21. The method of claim 17, wherein administering is via an ocular route, rectal route, topical route and/or intradermal route.
